(19) United States
(12) Patent Application Publication
(10) Pub. No.: US 2004/0210183 A1
(43) Pub. Date: Oct. 21, 2004

(54) Syringe barrel and syringe comprising same

(76) Inventor: Dieter Schulz, Muhlheim (DE)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 10/419,046

(22) Filed: Apr. 17, 2003

(51) Int. Cl. A61N 1/30

(52) U.S. Cl. 604/20

(57) ABSTRACT

A syringe for veterinary or human medicine, the syringe comprising a syringe barrel that receives a piston pressing on a liquid to be injected and comprises a wall having UV radiation-absorbing or UV radiation-reflecting properties. Thus, liquid with which the syringe is charged is protected from detrimental influences of UV radiation.

### BACKGROUND OF THE INVENTION

The invention relates to a syringe barrel for a syringe for veterinary or human medicine, which syringe barrel is provided to receive a piston pressing on a liquid to be injected and comprises a wall. The invention further relates to a syringe comprising such syringe barrel.

In syringes for veterinary or human medicine, syringe barrels are usually transparent, allowing to see, during use, whether and with how much liquid the syringe is charged. For ease of manufacture, the syringe barrels are, in most cases, made of one single material, usually by injection molding from plastics.

Particularly in the case of reusable syringes, high demands are made on robustness. This applies, to a greater extent, to syringes for veterinary medicine, which are capable of giving off a predetermined single dose from a larger volume of the syringe barrel upon one actuation, i.e. so-called repeater syringes.

Depending on the particular application, such syringes may require the use of a syringe barrel having as large a volume as possible, thus allowing to effect as many single injections as possible after one single charging operation. Then, the charged liquid, for example a pharmaceutical or any other preparation to be administered, must permit charging in a large volume and distribution as single injections over a longer period of time.

It is an object of the invention to provide a syringe barrel as well as a syringe, allowing to keep prepared, over a longer period of time, also larger quantities of liquid to be discharged in injections.

### SUMMARY OF THE INVENTION

According to the invention, in a syringe barrel for a syringe for veterinary or human medicine, which syringe barrel is provided to receive a piston pressing on a liquid to be injected and comprises a wall, this object is achieved in that the material of the wall has UV light-absorbing or UV light-reflecting properties. The object is further achieved by a syringe comprising such syringe barrel.

The design of the syringe barrel according to the invention protects liquid, once charged, from detrimental UV radiation, thus allowing a longer possible storage time of charged liquid. This is advantageous, in particular, if a syringe equipped with the syringe barrel according to the invention is to be used outdoors, which is often the case in veterinary medicine. Mass applications of liquids, for example mass vaccinations of sheep, pigs or cattle, are now possible out in the open field also in strong sunlight, without the risk of damaging charged liquid.

In order to determine with certainty whether or with how much liquid the syringe barrel has been charged, an embodiment is advantageous according to which the wall of the syringe barrel is transparent to light (i.e. to visible radiation), although not necessarily colorless. In order to accordingly achieve the aforementioned transparent properties, the material of the wall of the syringe barrel advantageously has spectrally selective absorbing properties, i.e. it absorbs UV radiation, but, on the other hand, if possible, transmits visible radiation, i.e. light. In many cases, the syringe barrel will be amber colored when this object is achieved.

For particularly good protection of charged liquid against damaging UV radiation, the wall of the syringe barrel preferably has a degree of absorption of at least 20% or higher, at a wavelength of from 350 to 380 or even 400 nm. A value of 30% is particularly preferred, since it offers better protection. A further increased degree of absorption of 40 or 50% further improves protection against damaging UV radiation.

A syringe barrel which can be manufactured by injection molding is favorable from the point of view of easy manufacture. Therefore, a plastic material which can be injection-molded is preferred as material for the syringe barrel. A class of plastic materials which, on the one hand, can be injection-molded, and, on the other hand, exhibits the desired absorption properties, is polyphenylsulfone (PPSU). Therefore, the syringe barrel preferably comprises this material or is even made completely thereof, for example by injection molding, at least in the area of the wall. Of course, other materials may also be used, such as glass or polycarbonate, which are inherently clearly transparent, but which may be provided with the desired UV radiation-absorbing or UV radiation-reflecting properties by adding suitable substances or coatings.

Polyphenylsulfone has further turned out to be advantageous, since it is, on the one hand, very stable against chemically aggressive substances or acids, for example, vitamin A acid. On the other hand, the shrinkage behavior of polyphenolsulfone during manufacture is nearly identical with the properties of polycarbonate, so that the manufacture of a syringe barrel, hitherto made of polycarbonate, can be easily changed to polyphenylsulfone, without having to modify injection molding tools or specified dimensions.

UV radiation can reach liquid with which the syringe barrel is charged primarily through the wall of the syringe barrel. Although it is possible, and sometimes also favorable from the point of view of costs, to manufacture the entire syringe barrel, including the injection needle attachment, from the same material as the barrel wall, so that the syringe barrel, as a whole, exhibits UV radiation-absorbing or UV radiation-reflecting properties, it is often preferable, for mechanical reasons, to manufacture a receiving part for an injection syringe from a different material. This applies, in particular, to injection syringes provided with standardized or widely used injection needle locking mechanisms, for example a Luer lock mechanism. For this purpose, metallic receiving parts are preferred, in particular in the case of reusable syringes, for reasons of mechanical stability. According to one embodiment of the invention, the syringe barrel, therefore, preferably comprises a receiving part for an injection needle at one end thereof.

At the other end, the syringe barrel may be inserted into a manipulating unit, which serves to hold, support or actuate the syringe, in particular via a screw connection. Such syringe barrel also has the advantage that syringes equipped with conventional syringe barrels may be subsequently fitted accordingly.

Of course, the syringe barrel according to the invention is suitable, in particular, also for disposable syringes. In this case, a tip for fitting an injection needle as well as corresponding manipulating elements, such as handles, are advantageously formed directly on the syringe barrel.

### DESCRIPTION OF THE FIGURES

The invention is explained in more detail below, by way of example and with reference to the drawings, wherein:

FIG. 1 shows a perspective view of a veterinary syringe;

FIG. 2 shows a vertical, longitudinal section of the veterinary syringe, and

FIG. 3 shows spectral absorption curves for the material of the syringe barrel of the veterinary syringe of FIG. 1.

### DETAILED DESCRIPTION OF PREFERRED EMBODIMENTS

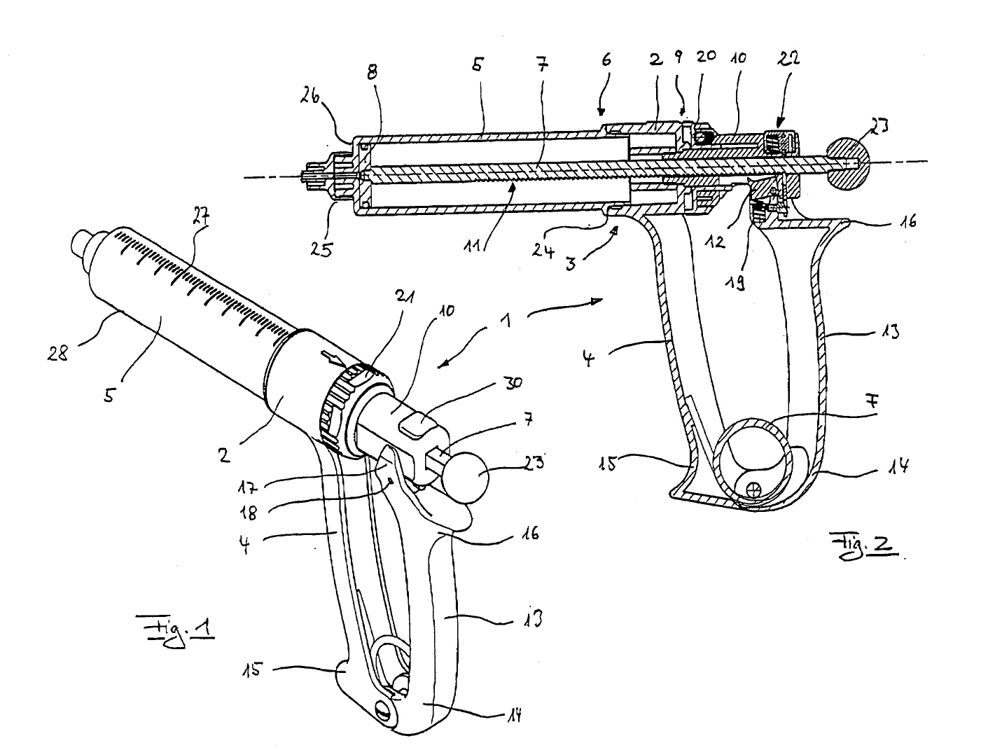

FIG. 1 shows a veterinary syringe 1, which comprises a base body 2 having a handle 4 arranged at the bottom surface 3 thereof, a syringe barrel 5, which is inserted at the front 6 of the base body 2 and into which a piston rod 7 comprising a piston 8 protrudes. The piston rod 7 is held, in a lengthwise slidable manner, in a guiding body 10 arranged on the back 9 of the base body 2. At its bottom surface, the piston rod 7 is formed as a rack 11, with which a pawl 12 may engage, said pawl being drivable by an operating lever 13. The operating lever 13 has a first end 14 thereof pivotably attached to the free end 15 of the handle 4 and has its second end 16 guided on the guiding body 10 longitudinally of the veterinary syringe 1. A spring F urges the operating lever 13 away from the handle 4. In the region of the guiding body 10, the second end 16 of the operating lever 13 is formed as a fork 17 which laterally embraces the guiding body 10. In this fork 17, there is provided a shaft 18, which is supported horizontally and transversely of the direction of movement of the piston rod 7, on which shaft 18 the pawl 12 is pivotably supported and is urged against the rack 11 by a spring 19 provided in the operating lever 13.

If the operating lever 13 is moved forward in a direction toward the handle 4 as far as a stop, the piston rod 7 is moved into the syringe barrel 5. Therefore, the length of movement of the operating lever 13 may be adjusted via a stroke-limiting device 20, so as to allow dosage of the quantity to be injected. The stroke-limiting device 20 is integrated into the guiding body 10 and comprises an adjusting ring 21, which is arranged between the back 9 of the base body 2, on the one hand, and the guiding body 10, on the other hand, and is rotatable, said adjusting ring 21 being adjustable to individual dosage levels and limiting the length of movement of the operating lever 13 for each actuation using suitable mechanics.

At the rear end of the guiding body 10, there is provided a locking device 22 for locking the rack 7 against inadvertent withdrawal of the piston rod 7. Further, at the end of the piston rod 7, there is provided a knob 23 by which the piston 8 may be moved into its starting position again and, thus, the syringe barrel 5 may be refilled with liquid.

The syringe barrel 5 is sealingly inserted into the handle 4 via a screw connection 24, so that the feed of the piston 8, which contacts the inside of the syringe barrel 5 in a sealing manner and presses on charged liquid, ejects said liquid from the syringe. At its needle-side end 26 the syringe barrel 5 has a suitable receiving part 25 for an injection needle.

The syringe barrel 5 is suitably transparent to light, so that it is easily verifiable, by means of a scale 27 provided on the wall 28 of the syringe barrel 5, how much liquid has been drawn up into the syringe.

Moreover, the wall 28 of the syringe barrel 5 has UV radiation-absorbing properties so that liquid with which the syringe barrel 5 is charged is protected against detrimental influences of UV radiation.

The UV radiation-absorbing properties of the wall 28 are achieved by the syringe barrel 5 being manufactured from polyphenylsulfone. For this purpose, in the described embodiment use is made of a material from Solvay Advanced Polymers, Alpharetta, Ga., USA, which material is sold under the trade name Radel R-5000 NT and is an injection-moldable, amber colored polyphenylsulfone resin. This material was dried for 2.5 hours at about 150° C. prior to injection molding and then injection molded in a mold at a temperature of from 360 to 390°, with the temperature of the mold ranging from 140 to 165°.

FIG. 3 shows the transmission properties of the wall 28 of the syringe barrel 5, with a percentage of transmission T being plotted as a function of the wavelength λ indicated in nm. As is evident therefrom, the wall has a degree of absorption of over 60% in a wavelength range of from 350 to 380 nm. The degree of absorption decreases as the wavelength increases, i.e. the degree of transmission increases. At a wavelength of 400 nm, a degree of transmission of about 40% is exceeded. The characteristics of the degree of transmission of the wall of the syringe barrel represented in FIG. 3 result in the amber color of the barrel. Of course, other colors are possible by adding dyes, for example, a gray syringe barrel may be obtained by adding a dark dye at a concentration of about 3%.

Shrinkage of polyphenylsulfone after molding is about 0.7% and is thus identical with that of polycarbonate. Therefore, the manufacture of the syringe barrel 5 can involve the use of injection molds designed for polycarbonate syringe barrels. Due to the manufacture of the syringe cylinder using an injection molding process, the entire syringe barrel 5 consists of the injectable plastic material, so that the UV radiation-absorbing properties are present throughout the syringe barrel.

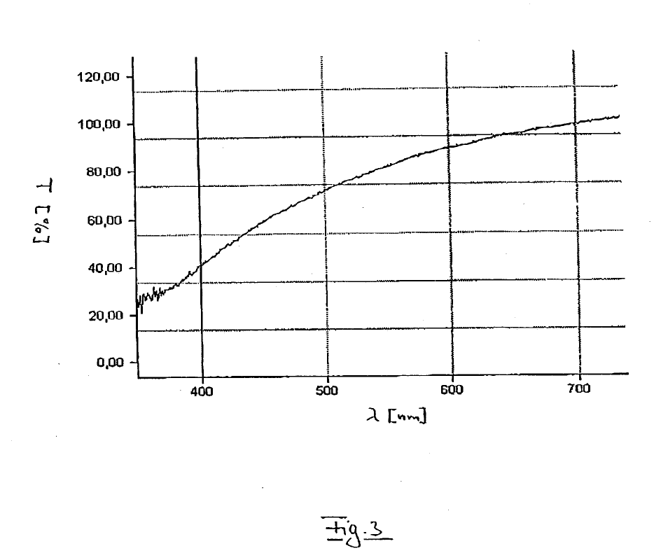

The knob 23 of the syringe of FIGS. 1 and 2 is removably fitted on the end of the piston rod 11, with a screw connection being used in the example of FIG. 2. Said knob is thus easily replaceable. Differently colored knobs are provided for the syringe, thus allowing an easy distinction between otherwise outwardly similar syringes for different applications. The user may fill syringes with different liquids, for example pharmaceuticals, and associate a differently colored knob 23 with each liquid. This is particularly advantageous, since the UV radiation-absorbing syringe barrel 5 now allows to charge a plurality of liquids and to use syringes prepared in this manner over a longer period of time. In this case, the differentiation by differently colored knobs 23 prevents any risk of confusion.

The screw connection 24 between the syringe barrel 5 and the handle 4 allows easy removal of the syringe barrel from the handle for cleaning. In connection with the thermally very stable polyphenolsulfone material of the syringe barrel 5, full autoclavability is achieved.

I claim:

1. A syringe barrel for a syringe for veterinary or human medicine and for receiving a piston pressing on a liquid to be injected, said syringe barrel comprising a wall, wherein the material of the wall has UV radiation-absorbing or UV radiation-reflecting properties.

2. The syringe barrel as claimed in claim 1, wherein the wall has a degree of absorption of at least 20%, preferably at least 30%, at a wavelength of from 350 to 380 nm.

3. The syringe barrel as claimed in claim 2, wherein the wall has a degree of absorption of at least 40%, preferably at least 50%, at a wavelength of from 350 to 380 nm.

4. The syringe barrel as claimed in claim 1, wherein the material comprises polyphenylsulfone.

5. The syringe as claimed in claim 1, wherein the syringe barrel consists of an injection-moldable plastic material.

6. The syringe barrel as claimed in claim 1, wherein the syringe barrel comprises, at one end thereof, a receiving part for an injection needle.

7. A syringe for veterinary or human medicine for injection of liquids which comprises a syringe barrel as claimed in claim 1.

8. The syringe as claimed in claim 7, wherein the syringe barrel is inserted, in particular screwed, into a manipulating unit for holding, supporting or actuating the syringe.

9. The syringe as claimed in claim 7, characterized in that it is provided as a disposable syringe.

10. A syringe for medicinal use for injecting liquids, the syringe comprising a syringe barrel and a piston for pressing on the liquid to be injected, said syringe barrel comprising a wall, characterized in that the material of the wall is transparent and has UV radiation-absorbing or UV radiation-reflecting properties.

11. The syringe as claimed in claim 10, characterized in that the wall of the syringe barrel has a degree of absorption of at least 20%, preferably at least 30%, at a wavelength of from 350 to 380 nm.

12. The syringe as claimed in claim 10, characterized in that the wall of the syringe barrel has a degree of absorption of at least 40%, preferably at least 50%, at a wavelength of from 350 to 380 nm.

13. The syringe as claimed in claim 10, wherein the syringe barrel is comprised of polyphenylsulfone.

14. The syringe as claimed in claim 10 wherein in that the syringe barrel consists of an injection-moldable plastic material.

15. The syringe as claimed in claim 10, characterized in that the syringe barrel is inserted, in particular screwed, into a manipulating unit for holding, supporting or actuating the syringe.

16. The syringe as claimed in claim 10, characterized in that it is provided as a disposable syringe.

17. A method of manufacturing a syringe comprising the steps of
selecting a transparent UV resistant injection moldable plastic;
injection molding a syringe barrel from said plastic; and
assembling the syringe barrel into a syringe.

18. The method of claim 17 further comprising the step of selecting polyphenylsulfone

19. The method of claim 17 further comprising the step of selecting a transparent plastic with a degree of absorption of at least 20%, preferably at least 30%, at a wavelength of from 350 to 380 nm.

20. The method of claim 17 further comprising the step of selecting a transparent plastic with a degree of absorption of at least 40%, preferably at least 50%, at a wavelength of from 350 to 380 nm.
